(19) United States
(12) Patent Application Publication
(10) Pub. No.: US 2016/0185732 A1
(43) Pub. Date: Jun. 30, 2016

(54) METHOD AND INDUSTRIAL PROCESS FOR CONTINUOUS SYNTHESIS OF DIFFERENT IONIC LIQUIDS

(71) Applicant: TULIPORT S.A.R.L., Luxemburg (LU)

(72) Inventor: Rainer Pommersheim, Mainz (DE)

(73) Assignee: Tuliport S.A.R.L., Luxemburg (LU)

(21) Appl. No.: 14/889,670

(22) PCT Filed: May 6, 2014
(86) PCT No.: PCT/EP2014/059150

§ 371 Date: Mar. 15, 2016

(30) Foreign Application Priority Data
May 7, 2013 (DE) 10 2013 007 733.8

(51) Int. Cl. C07D 233/58; B01J 19/24

(52) U.S. Cl. CPC C07D 233/58; B01J 19/24; B01J 2219/00182; B01J 2219/24

(57) ABSTRACT

Producing different ionic liquids industrially currently requires either operating separate plants or continually refitting existing plants. While this is not laborious, it is often not economical either. The problem the present invention addresses is that of describing a method and an industrial process by which different ionic liquids can be produced almost synchronously in a unitary production sequence. This is done either by using a chemical intermediate both relatively simple to prepare and readily convertible into multiple products, or by preparing the desired products in the same industrial process by direct synthesis from the appropriate starting materials. The method described here allows the synthesis of different ionic liquids in excellent yields and grades in a unitary production operation.

The invention relates to a method and an industrial process for producing ionic liquids in a continuous method. The entirety is conceived so that different ionic liquids can be obtained nearly simultaneously. For this purpose, a chemical intermediate stage is used, which not only can be produced relatively simply, but rather can also be converted without great effort into a variety of products. The method described here enables the synthesis of different ionic liquids in outstanding yields and qualities in a uniform production process.

The term ionic liquids is understood as liquids which are exclusively made of ions. These are molten salts of organic compounds or eutectic mixtures of organic and inorganic salts in this case.

Ionic liquids themselves have an array of outstanding properties: they are nonvolatile (negligible vapor pressure, like salts), are only combustible with difficulty, and are thermally stable (depending on the selected liquid, up to greater than 300° C.). Most ionic liquids are non-toxic.

To provide a delimitation from classic molten salts, which have a high melting point and are strongly corrosive, the melting point of ionic liquids is fixed by definition at temperatures of less than 100° C. Large organic cations are typical for the structure of ionic liquids, more precisely these are onium ions in this case, which are often formed by nitrogen or phosphorus centers and alkyl groups.

These cations can be combined with a variety of organic or inorganic anions. The modular synthesis of such liquids enables the physical and chemical properties to be varied in a targeted manner in a broad range by way of suitable combination of cations and anions. Primarily, stability and other fundamental physical properties of the ionic liquid are influenced by the selection of the cation, while the selection of the anion determines the chemistry and functionality. The possibility of adapting relevant chemical and also physical properties step-by-step enables the development of novel ionic liquids which meet the requirements of specific tasks by 100%.

Ionic liquids display interesting properties as an alternative solvent in chemical and biocatalytic reactions: the lack of volatility thereof offers advantages in the method technology. In addition, their extraordinary solubility properties open up new possibilities in chemical syntheses. The ionic liquids themselves can be easily reclaimed after use in most cases and used again, which additionally increases the efficiency of the chemical processes.

Several large-scale industrial applications of ionic liquids are listed hereafter as examples:

### Chemical Industry

BASF is using ionic liquids in an industrial scale for the first time worldwide in their new BASIL (biphasic acid scavenging utilizing ionic liquids) method. Harmful acids which would decompose the final product are removed from the process here by way of the use of ionic liquids. In this manner, the yield of the method can be increased significantly in relation to the conventional methods.

Cellulose is a raw material which plays a central role not only in the paper and fiber industry. Thanks to their extraordinary solubility properties for biopolymers, ionic liquids, for example, imidazolium acetate, open up completely new possibilities for novel methods and products. Other, toxic solvents can be omitted by way of the use thereof.

In electrochemical processes, for example, aluminum plating, ionic liquids which contain, for example, an imidazolium cation and a chloride anion, offer significant advantages as an electrolyte in relation to conventional materials.

Ionic liquids, for example, imidazolium sulfate, are very well suitable as antistatic agents for plastics.

### Petroleum Chemistry

The average sulfur content in crude oil has increased significantly in recent decades. This is not least because more and more deposits of lower crude oil quality are being exploited. Current diesel and gasoline engines require fuels having an extremely low sulfur content, however. Crude oil also can no longer be processed in the refineries from a specific sulfur content. Therefore, great efforts are taken during the preparation of the crude oil to reduce the sulfur content. This requires complicated chemical method steps, sometimes having a high environmental strain. With the aid of ionic liquids, for example, methyl imidazonium (MIM) derivatives, sulfur can be removed from the crude oil by simple washing, as shown by the work of multiple national and international research groups.

### Electrochemistry

Because of their ionic character, ionic liquids have a large potential as an electrolyte in electrochemical stores such as batteries, capacitors, etc. Although several of them have been used in so-called lithium-ion batteries for years, novel compounds and production methods for ionic liquids for this intended application are always being sought feverishly worldwide.

### Photovoltaics

So-called DSSC photovoltaics modules are a new generation of cells, which are very promising. They function according to a principle similar to plant photosynthesis and can also still deliver relatively high energy yields in the event of poor or diffuse light. Special electrolytes having very particular properties are required to enable the charge exchange inside the DSSC cells. Ionic liquids fulfill these requirements. The development of the DSSC cells is therefore closely linked to the ionic liquids.

Against this background, it is not surprising that both numerous synthesis possibilities and also more and more applications for ionic liquids have been described in the literature of recent years. Several of these are summarized hereafter as examples:

In published application DE 10 2005 025 531 A1, for example, different ionic liquids of low viscosity and high electrochemical stability are described, which are primarily intended for electrochemical applications. Several synthetic routes for how these compounds can be produced in the laboratory are also disclosed.

Patent application DE 103 19 465 A1 is concerned with the production in a laboratory scale of ionic liquids having alkyl sulfates or functionalized alkyl sulfates as the anion. These compounds are of substantial industrial significance as halogen-free solvents, extraction agents, and thermal carriers.

A laboratory method for producing ionic liquids having halogen-containing anion is the subject matter of patent application EP 1182196 A1.

Alkyl ammonium salts as a cation of an ionic liquid and the production method thereof are described in application GB 2444614 A1.

Ionic liquids having alkyl sulfates as anions, and a laboratory method for the production thereof, are the subject matter of application US 2008 033178 A1.

A large problem in the production of ionic liquids in large quantities is the control of the temperature during the reaction process. To bring the starting materials to reaction, heat must first be supplied to the system. If the reaction has been started, but it runs strongly exothermically, efficient dissipation of the resulting heat output of the system is required.

Application DE 102008032595 A1 is extensively concerned with these problems and describes an industrial method in which both the required activation heat and also the resulting reaction heat are controlled by way of the use of a suitable solvent.

All of these citations show how manifold the ionic compositions and the synthesis possibilities are for ionic liquids. The described methods are practical in the laboratory scale and are also suitable for industrial production in individual cases. However, they reach their limits when the intention is to produce multiple substances in an industrial scale in a uniform process.

An operation which wishes to offer different ionic liquids either has to operate different facilities or continuously refit existing facilities. This is not only complex but rather also not cost-effective in many cases.

Proceeding from this state of affairs, the present invention is based on the object of describing a method and an industrial process, using which different ionic liquids can be produced nearly simultaneously in a uniform production sequence.

The basic idea of the method is accordingly, in a continuous method, to synthesize an intermediate stage, which can be converted using simple, conventional means into the different final products, i.e., ionic liquids.

Such a suitable intermediate stage can be so-called imidazolium-based carboxylates. Methods for the production thereof are known in the literature. Thus, for example, in Green Process Synth (2012): 261-267, a laboratory process is described, in which N-methylimidazole is alkylated with dimethyl carbonate. If one proceeds from other alkylation reagents, as described in Chemical Engineering Journal 163 (2010) for 29-437, for example, one obtains the corresponding halogenides or sulfates of the methylimidazole.

Such intermediate stages can subsequently be converted by admixing with acids, for example, acetic acid, for example, into the corresponding imidazolium acetate, by the reaction with hydrochloric acid into imidazolium chloride, or with nitric acid into the corresponding nitrate, i.e., different ionic liquids which are based on the imidazolium cation.

Since intermediate stages, which are the same or are comparable in synthesis technology, are always used as the starting material, the different ionic liquids can be produced within the same industrial processes.

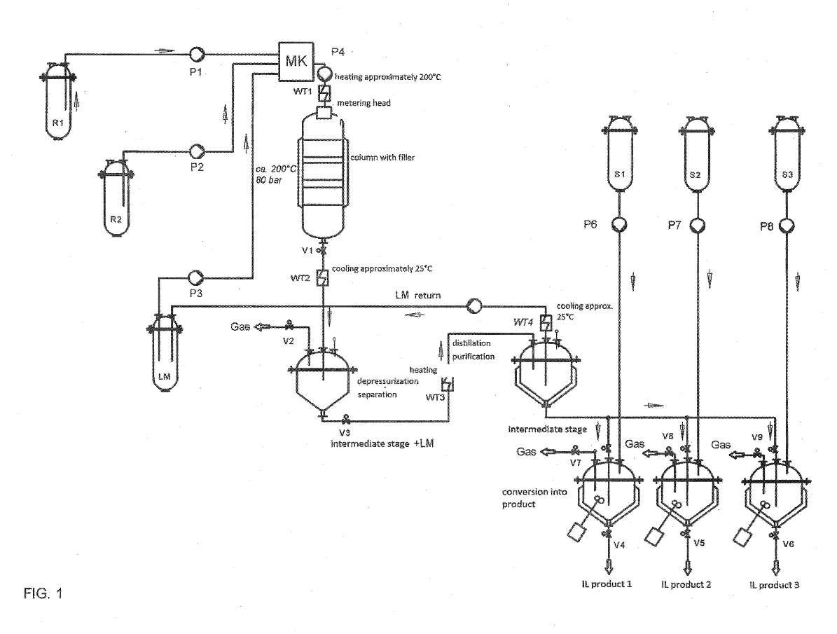

Examples of a possible industrial process for production of ionic liquids as described in the present invention is shown hereafter on the basis of the schematic diagram from FIG. 1:

### EXEMPLARY EMBODIMENT 1

In the process from FIG. 1, the starting substances R1 (for example, dimethyl carbonate) and R2 (for example, methylimidazole), and a solvent (for example, methanol) are provided in corresponding containers and transported therefrom via the pumps [P1, P2, and P3] to a mixing chamber [MK]. The solvent has the task, inter alia, of keeping the reaction temperature within narrow boundaries. The mixture is discharged from the mixing chamber via the pump [P4] at the head of a continuously operated reactor under pressure (for example, p=80 bar). The mixture can be preheated beforehand. The task can be performed in this case by a suitable device, for example, an individual nozzle or by nozzle arrays in the form of drops, flowing liquid, or by spraying.

The continuously operated reactor is heated or cooled in a suitable manner, for example, externally or by elements from the interior, to be able to set the reaction temperature. The reactor can contain additional fittings, which enable a narrow dwell time distribution. A filler is located in the reactor, which consists either of conventional filler materials such as Raschig rings or the like. However, it can also contain substances which unfold a catalytic effect, for example, metal oxides. The temperature in the reactor is approximately 200° C., the pressure is approximately 80 bar.

The starting substances react during the passage through the reactor. In this case, either unreacted starting substances or the ionic liquid dissolve in the solvent which is used. After leaving the reactor via the valve [V1], the resulting intermediate product (for example, the methyl imidazolium carboxylate) is cooled together with the solvent in the heat exchanger [WT2] to room temperature and conducted into the separating unit. Depressurization occurs therein, and the gas arising during the reaction (for example, CO₂) is removed.

From the separating unit, the mixture is supplied to a distillation, where the intermediate product (intermediate stage) is separated from the solvent. The solvent is recirculated via the pump [P6].

The mixture can be preheated beforehand via the heat exchanger [WT3]. WT3 can be coupled to WT2 so that reclamation is achieved.

The intermediate stage thus obtain subsequently reacts to form the desired final product. This is performed in one or, as shown in FIG. 1, in multiple reaction vessels. In these containers, the intermediate stage is admixed with a suitable acid, whereby CO₂ and the desired final product result from the intermediate stage. Different acids result in different products. Thus, for example, if acetic acid is added, the methyl imidazolium acetate results, the corresponding chloride results with hydrochloric acid, the corresponding nitrate with nitric acid, etc. The acids (S1, S2, S3, etc.) are supplied via the metering pumps [P6], [P7], [P8] to the respective reactor. The resulting ionic liquids (IL product 1, IL product 2, IL product 3, etc.) are supplied via the valves [V4], [V5], and [V6] to subsequent processing or purification.

### EXEMPLARY EMBODIMENT 2

The process from FIG. 1 can also be used for the purpose of producing individual, specific ionic liquids directly, i.e., not via an intermediate stage. For this purpose, different starting materials have to be used than in Example 1.

The starting substances R1 (for example, diethyl sulfate) and R2 (for example, methyl imidazole) are transported via the pumps [P1 and P2] to a mixing chamber [MK]. The mixing chamber can be brought to the starting temperature via a cooling or heating device. A suitable quantity of a solvent (for example, toluene, ethyl acetate, etc.) is continuously supplied to this mixture via the pump [P3]. The solvent has the task of keeping the reaction temperature in narrow boundaries. In this case, either the ionic liquid to be formed or unreacted starting substances are to be soluble in the selected solvent. The components R1, R2 and the solvent LM are discharged via the pump [P4] at the head of a continuously operated reactor. The task can be performed in this case by a suitable device, for example, an individual nozzle or by nozzle arrays in the form of drops, flowing liquid, or by spraying.

The continuously operated reactor is heated or cooled in a suitable manner, for example, externally or by elements from the inside, to be able to set the reaction temperature. The reactor can contain additional fittings, which enable a narrow dwell time distribution, or can unfold a catalytic effect. Depending on the requirement, a temperature gradient can additionally be set in the reactor.

The starting substances react during the passage through the reactor. In this case, either unreacted starting substances or the ionic liquid dissolve in the solvent which is used. After leaving the reactor via the valve [V1], the resulting liquid phases are separated in the separating unit. The predominant part of the solvent, which forms a second phase with the product is recirculated, for example, via the gas fitting and via an additional pump to be installed, into the solvent container. The final product is already supplied to the distillation via [V3] in this case, where it is purified of the solvent residues. The final product is thus already obtained at the outlet of the distilling unit. If the starting substances from this example are used, this is methyl imidazolium diethyl sulfate.

1. A method and an industrial process for synthesis of ionic liquids, in which, in a uniform production sequence, different ionic liquids can be produced, characterized in that the synthesis runs via a chemical intermediate stage, which can be converted using conventional means into different final products.

2. The method and industrial process for synthesis of ionic liquids according to claim 1, characterized in that the chemical intermediate stage can itself be used as the final product in the event of a suitable selection of the starting substances.

3. The method and industrial process for synthesis of ionic liquids according to claim 1, characterized in that the chemical intermediate stage is, for example, a carboxylate, a carbonate, or a comparable compound.

4. The method and industrial process for synthesis of ionic liquids according to claim 1, characterized in that the chemical intermediate stage can be converted into the final product by adding an organic or inorganic acid.

5. The method and industrial process for synthesis of ionic liquids according to claim 1, characterized in that the added solvent also assumes the heat transport, or sets the reaction temperature by way of its vaporization temperature, i.e., is used for temperature control.

6. The method and industrial process for synthesis of ionic liquids according to claim 1, characterized in that the reactor has fittings or a filler which also unfolds a catalytic effect.

7. The method and industrial process for synthesis of ionic liquids according to claim 1, characterized in that the fittings or the filler in the reactor consist of elements which consist, for example, of metal, glass, carbon, or a metal oxide, or as are used, for example, in distillation columns in the chemical industry.

8. The method and industrial process for synthesis of ionic liquids according to claim 1, characterized in that open-chain or cyclic compounds are used as the starting substances, which results upon the reaction with a suitable alkylation agent in ionic liquids of the type A⁺B⁻, wherein A is, for example, a material from the class of amines or imines, for example, pyridine, piperidine, imidazole, and B is, for example, a material from the class of halogen alkanes or substituted halogen alkanes, for example, diethyl sulfate, dimethyl carbonate, etc.

9. A facility according to claim 1, which operates according to a method according to claim 1, characterized in that it has one or more of the following main components:
storage container R1 for the starting material of the type A,
storage container R2 for the starting material of the type B,
storage container LM for the solvent,
storage containers S1, S2, etc. for acids,
metering pumps and/or pumps P1; P2; P3; P4; P5, etc.,
mixing chamber MK,
reactor having drip or spraying unit and filler
valves V1; V2, etc.,
separating unit,
distillation unit.

10. The facility according to claim 9, characterized in that it operates according to FIG. 1 and/or the components thereof are arranged and/or connected to one another according to FIG. 1 or in a similar manner.
